(19) United States
(12) Patent Application Publication
(10) Pub. No.: US 2025/0195076 A1
(43) Pub. Date: Jun. 19, 2025

(54) Catheter for the Delivery of Y90 to an Individual

(71) Applicant: The Regents of the University of California, Oakland, CA (US)

(72) Inventor: Jaehoon Shin, San Francisco, CA (US)

(21) Appl. No.: 18/982,562

(22) Filed: Dec. 16, 2024

Related U.S. Application Data
(60) Provisional application No. 63/611,643, filed on Dec. 18, 2023.

(51) Int. Cl. A61B 17/12; A61B 17/00; A61M 25/00; A61M 39/06; A61M 39/10

(52) U.S. Cl. CPC A61B 17/12109; A61B 17/00234; A61B 17/12136; A61B 17/1214; A61B 17/12159; A61M 25/0026; A61M 25/0097; A61M 39/0613; A61M 39/10; A61B 2017/00238; A61B 2017/00305; A61B 2017/1205; A61M 2039/1077; A61M 2202/0007; A61M 2202/049; A61M 2202/06; A61M 2210/12

(57) ABSTRACT

Methods, systems, and devices for the delivery of a therapeutic using a catheter are provided. Many embodiments are directed to a catheter with one or more lumens that can be used to deliver a therapeutic to a tumor. Many embodiments include an occlusion mechanism that can block off downstream vasculature to prevent damage to normal or healthy tissue caused by the therapeutic. Numerous embodiments are used to deliver a bolus of Yttrium-90 microspheres to a tumor. Also provided are methods of using such catheters.

### CROSS-REFERENCE TO RELATED APPLICATION

This application claims benefit under 35 U.S.C. § 119(e) of provisional application 63/611,643, filed Dec. 18, 2023, which application is hereby incorporated by reference in its entirety.

### FIELD OF THE INVENTION

The present invention relates to the delivery of beads of Y90 to an individual suffering from cancer, more specifically, methods of delivering Y90 to an individual that occlude downstream vasculature to prevent damage to healthy tissue and catheters to accomplish the same.

### BACKGROUND

Yttrium-90 (Y90) radioembolization is a medical procedure designed to deliver beads that emit high-dose radiation directly to cancer supplying vessels. The goal of Y90 radioembolization is to induce complete necrosis of target tissues, similar to what is achieved with more invasive surgical resections. Typically, a catheter is maneuvered into a position near blood vessels supplying a tumor. Once in position, the Y90-loaded microspheres (or beads) are injected into these blood vessels. The microspheres become lodged in the small blood vessels near the tumors, delivering localized radiation.

A significant problem in Y90 radioembolization is minimizing delivery of the microspheres to surrounding normal tissues. Current approaches involve delivering Y90 beads as selectively as possible to tumor-feeding arteries. However, it is common that tumor supplying arteries are too small and tortuous, thus cannot be selectively catheterized. In such situations, beads are delivered less selectively, which has shortcomings, including a less effective radiation delivery to tumors and a sacrifice of a large amount of normal surrounding tissues. Thus, there is a significant need in the field for methods, systems, and devices that allow for Y90 radioembolization without sacrificing healthy or normal tissue.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

Methods, systems, and devices for the delivery of a therapeutic using a catheter are provided. Many embodiments are directed to a catheter with one or more lumens that can be used to deliver a therapeutic to a tumor. Many embodiments include an occlusion mechanism that can block off downstream vasculature to prevent damage to normal or healthy tissue caused by the therapeutic. Numerous embodiments are used to deliver a bolus of Yttrium-90 microspheres to a tumor. Also provided are methods of using such catheters]

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a schematic of a therapeutic delivery catheter in accordance with various embodiments.

FIGS. 2A-2E illustrate various configurations of lumens and catheter lumen strategies in accordance with various embodiments.

FIG. 3A-3C illustrate positioning of a catheter to achieve flow diversion and distal tissue protection by deploying an endovascular occlusion device (coils, balloons, or plugs) as in FIGS. 3A and B, or by perfusing fluid to induce reflux as in FIG. 3C in accordance with various embodiments.

FIGS. 4A-4C illustrate schematics of a Y-shaped adapter and its positioning in accordance with various embodiments.

FIGS. 5A-5B illustrate positioning a catheter with an occluding balloon into as vascular simulant in accordance with various embodiments.

FIGS. 6A-6B illustrate fluid flow through a vascular simulant when a catheter and a deflated occluding balloon are positioned in accordance with various embodiments.

FIGS. 7A-7B illustrate how inflation of the balloon prevents downstream blood flow to normal tissue through a vascular simulant, and all flow moves through tumor vasculature in accordance with various embodiments.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

Yttrium-90 (Y90) is a radioactive isotope that emits beta radiation to destroy cancer cells. Typically, Y90 is incorporated into tiny microspheres, which are injected into the blood vessels supplying tumors. Y90 radioembolization is primarily used to treat liver cancer, particularly for primary hepatocellular carcinoma (HCC) or metastatic liver tumors. The procedure generally involves inserting a catheter into the hepatic artery, the main blood vessel that supplies liver. Because Y90 microspheres result in complete necrosis of their target tissues, Y90 beads should be delivered as selectively as possible to the tumor vasculature. However, it is often impossible to selectively catheterize tumor supplying vessels due to their small size, necessitating delivery in more proximal vessels that also supply normal tissues, which risks damaging large volume of healthy tissue. One current strategy to increase selectivity involves injecting a biodegradable embolic material (e.g., gel foam) to block vessels supplying normal tissue, thereby preventing Y90 microsphere delivery to those areas. However, the embolic material can dislodge and inadvertently block the tumor-supplying vasculature instead. Once dislodgement occurs, the procedure must be terminated because the appropriate dose cannot be delivered to the tumor. The Y90 microspheres prepared for the treatment are wasted, as they are specifically calibrated for the patient on a particular day and thus cannot be used again. Another current strategy involves inserting two catheters-one for blocking the vessel supplying normal tissue, and the other for delivering therapeutics. This approach requires introduction of two separate catheters, thereby increasing the complexity of the case and the risk of vascular complications.

Many embodiments described herein provide for a catheter that can be used to occlude a downstream blood vessel to normal tissues and deliver Y90 microspheres to the tumor. In many instances, the occlusion is reversible and/or temporary.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

An “amount” as used herein refers to a quantity specified (e.g., high or low) or a number e.g., where the number is a level, such as a position on a real or imaginary scale of amount or quantity, or a concentration, such as, for example, a relative amount of a given substance contained within a solution or in a particular volume of space, e.g., the amount of solute per unit volume of solution.

The terms “bead,” “microbead,” and “microsphere” are used herein interchangeably and refer to a substantially spherical solid support. Such beads, microbeads, or microspheres may be constructed of any composition, including glass, metals, resins, etc. Certain beads may be embedded and/or coated with a therapeutic agent. The microspheres can be of any size that would work in the methods described herein, e.g., from about 10 μm to about 50 μm.

The terms “antegrade” and “retrograde” refer to flow of a fluid through a system, such that antegrade refers to the normal direction, while retrograde refers to a flow against the normal direction. In regards to blood flow, antegrade refers to the flow of blood from the heart through arteries, then veins, and returning to the heart, while retrograde refers to flow running opposite direction.

The terms “upstream” and “downstream” refer to positions based on antegrade flow within a system, such that an upstream position is located at an earlier position in antegrade flow, while a downstream position is located at a later position in antegrade flow.

The terms “proximal” and “distal” refer to positions relative to a reference position, such that proximal indicates a position closer to the reference position, while distal refers to a position further form the reference position.

The term “lumen” refers to the central, open space, or cavity within the walls of a tube. It is a hollow interior through which fluid, gas, or other substances may flow.

The term “occlusion” refers to a partial or complete obstruction or blockage of a passage, channel, or vessel. It can involve the restriction or closure of a lumen, preventing or limiting the normal flow of fluids, such as blood or air.

### Dual-Lumen Distal Protection Catheters

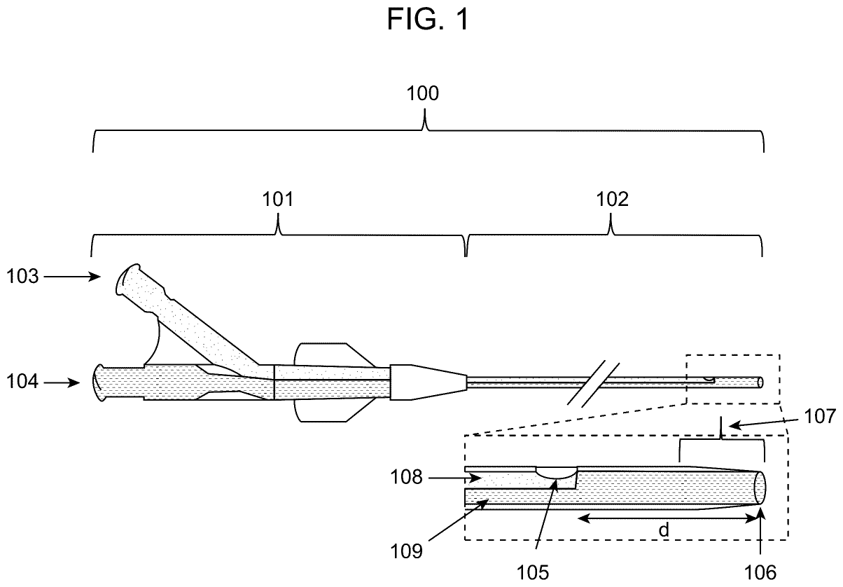

Many embodiments are directed to a dual-lumen catheter for selectively delivering a therapeutic to vessels supplying tumor, while also providing protection to the adjacent normal vessels. In many cases, the therapeutic is a bolus of Y90 microspheres. An example of catheters in accordance with these embodiments is illustrated in the schematic catheter 100 of FIG. 1. Catheter 100 includes a hub 101 and a catheter body 102. In many embodiments, the hub 101 includes a first port 103 and a second port 104. In the illustrated embodiment, the first port 103 is illustrated as a side hole port connected to and in fluid communication with a side hole 105 via a first lumen 108, where the side hole 105 and the first lumen 108 are formed by the catheter body 102. The second port 104, illustrated as an end hole port, is connected to and in fluid communication with an end hole 106 via a second lumen 109. In many embodiments, the catheter 100 (via the second port 104, the second lumen 109, and the end hole 106) is configured to accept a distal occlusion device, including (but not limited to) a coil, a plug, or a balloon. In many embodiments, the catheter 100 is configured to deliver a therapeutic via the first port 103, the first lumen 108, and the side hole 105).

Examples of deliverable therapeutics can include (but are not limited to) a chemotherapeutic, a small molecule, a large molecule, a biologic, an immunogenic, a radiotherapeutics (e.g., Y90 beads), any other applicable therapeutic, and combinations thereof. The catheter body 102 can be a generally cylindrical body. The catheter body 102 adjoins and is connected to the hub 101 at its proximal end. As noted above, the catheter body 102 can form the first lumen 108, the second lumen 109, the side hole 105, and the end hole 106. As illustrated, end hole 106 is formed at the distal end of the catheter body 102, and the side hole 105 is located at a distance d proximally from the distal end of the catheter body 102. Distance d can range from 0 cm (i.e., where the side hole 105 terminates at the distal end with end hole 106) to approximately 10 cm, such as 0 cm, approximately 1 cm, approximately 2 cm, approximately 3 cm, approximately 4 cm, approximately 5 cm, approximately 6 cm, approximately 7 cm, approximately 8 cm, approximately 9 cm, or approximately 10 cm.
In addition to being configured for an occlusion mechanism, the second lumen 109 and end hole 106 can be used for guiding wires to assist with positioning or navigating a catheter 100. In many embodiments, the distal end of the catheter body 102 forms a distal tip 107. In many of these embodiments, the distal tip 107 is tapered (i.e., the outer diameter at a proximal position is greater than the outer diameter at a distal position). This taper can assist catheter positioning by achieving better tracking through vessels over the guiding wires.

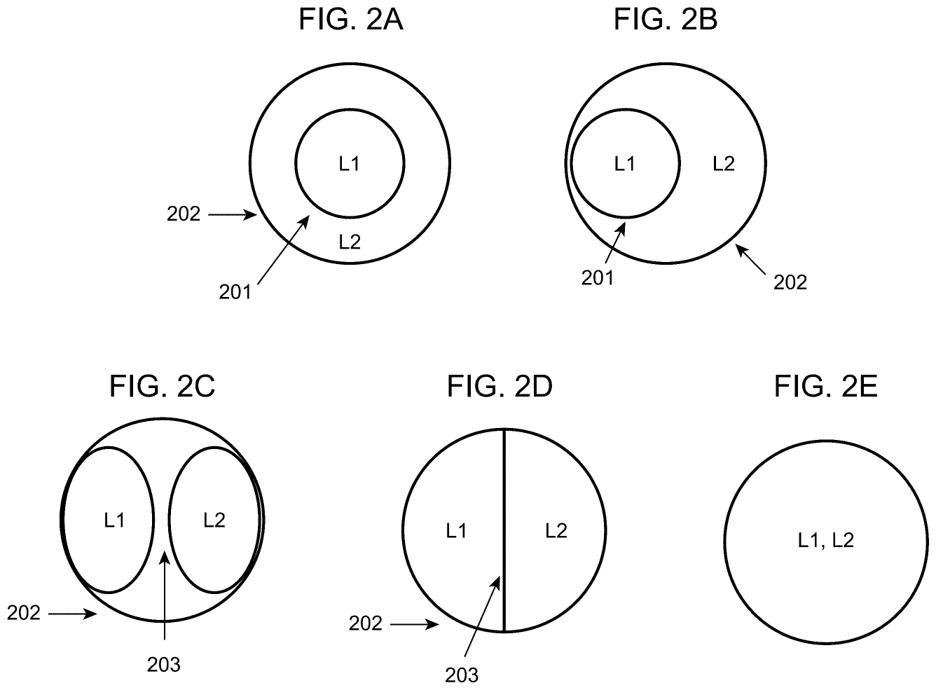

As noted above, many embodiments possess two lumens. FIGS. 2A-2F illustrate various exemplary cross sections of a catheter body 102 to form the two lumens. Using two lumens, a first lumen can be used for delivering therapeutics, while a second lumen can be used to hold and/or deliver an occlusion mechanism. FIG. 2A illustrates an example of concentric lumens, while FIG. 2B illustrates an example of off-centric lumens. In FIGS. 2A-2B, a first lumen (L1) is formed by an inner lumen wall 201, while a second lumen (L2) is formed by an outer lumen wall 202. In many instances, the outer lumen wall 202 is the catheter body (e.g., catheter body 102). In these examples, the inner lumen wall 201 forms a boundary between the first lumen L1 and the second lumen L2.

FIGS. 2C-2D illustrate examples of catheter body cross sections that include a septum 203 that forms the boundary between the first lumen L1 and the second lumen L2. In FIGS. 2C-2D, the septum 203 can bisect outer wall 202 and form the first lumen L1 and the second lumen L2 as generally round or ovular (FIG. 2C) or as hemispheric shapes (FIG. 2D). While FIG. 1 and FIGS. 2A-2D refer to a first lumen and a second lumen, the labeling as a “first” and “second” are merely to distinguish between different lumens. In other words, the first lumen illustrated in FIG. 1 may be a second lumen illustrated in one or more of FIGS. 2A-2D.

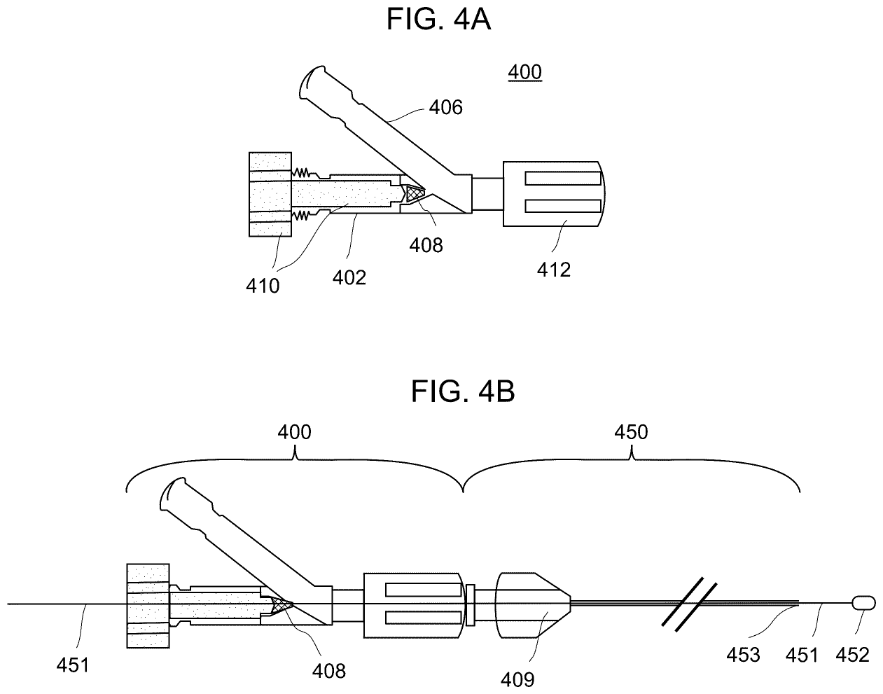

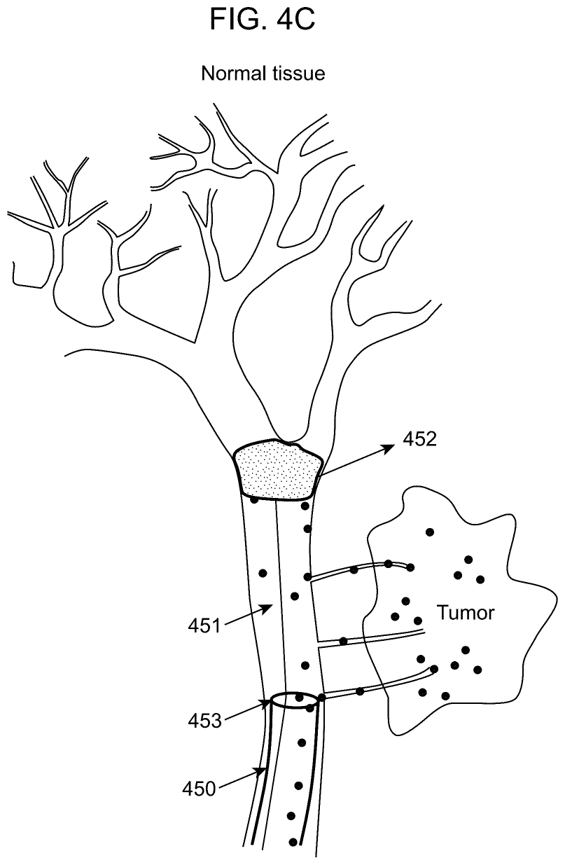

Alternative embodiments can utilize a single lumen catheter body, as illustrated in FIG. 2E. In a single lumen design, the lumen can possess a large enough size or cross sectional area to hold an occlusion mechanism as well as allow a therapeutic to move around the occlusion device. In such instances, traditional Y-adapters (e.g., Tuohy-Borst adapters) may allow for backflow or fluid stagnation of a therapeutic due to a dead-space within the adapter. Thus, to improve single-lumen delivery of a therapeutic, many embodiments implement a Dead-Space-Free Y-adapter, such as illustrated in FIGS. 4A-4B and described below.

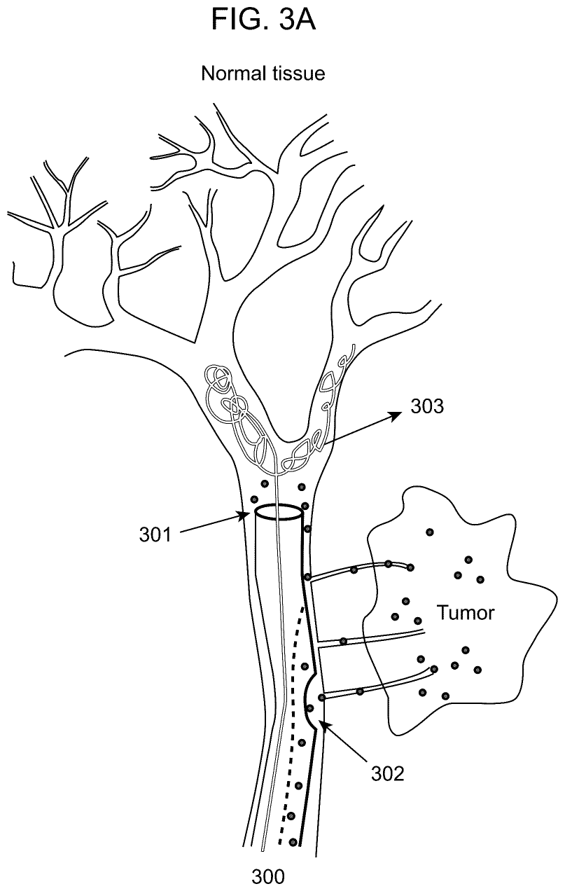

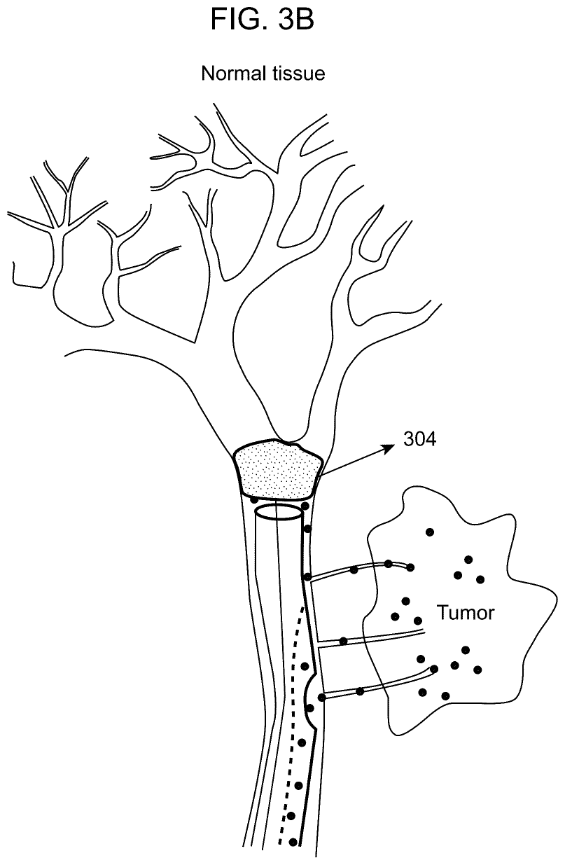

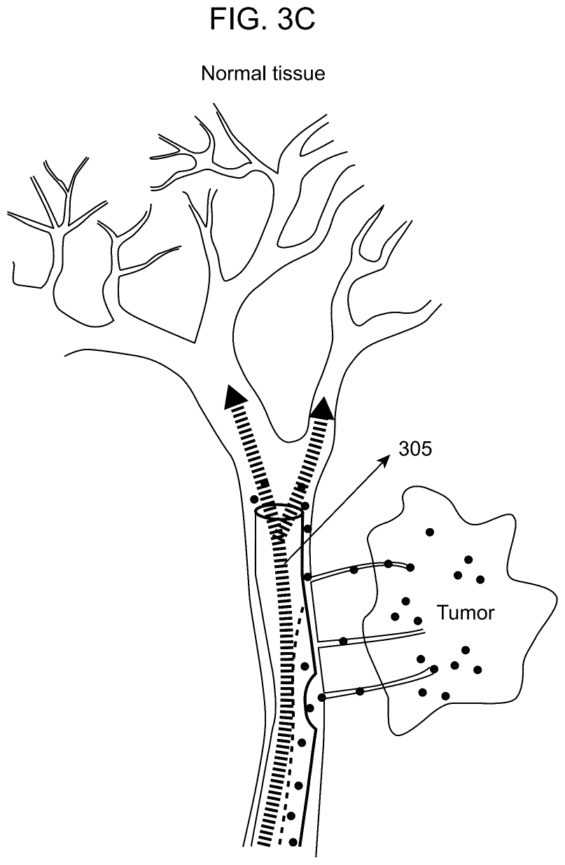

Many embodiments include an occlusion mechanism capable of temporarily and reversibly blocking off downstream vasculature (e.g., vasculature to normal or healthy tissue). Referring to FIG. 1, the occlusion mechanism can be introduced through the second port 104 and deployed distal to the end hole 106. This configuration allows for flow diversion away from the normal tissue, thereby increasing flow into tumor vasculature, as shown in the examples of FIGS. 3A-3C. As shown in FIGS. 3A-3C, the catheter 300 is positioned such that the end hole 301 is located at the normal tissue-supplying vasculature distal to the tumor-supplying vasculature, and the side hole 302 is positioned in the vicinity of the tumor supplying vasculature. Various strategies can be used as an occlusion mechanism, including an endovascular coil (FIG. 3A, 303), a balloon or a plug (FIG. 3B, 304), and/or any other mechanism suitable for occluding a blood vessel. In many instances, the occlusion mechanism is connected to a long shaft stiff enough to push the occlusion device through and outside of the catheter lumen and to retrieve the occlusion device back into the end hole and pull it back through the catheter. The occlusion device is thus temporary or reversible, so that they can be removed at the end of the delivery of therapeutics without permanently occluding vessel supplying normal tissue.

Additionally or alternatively, certain embodiments, as shown in FIG. 3C induce a localized reflux of blood flow around the distal tip of the catheter by continuously perfusing fluid (e.g., plasma, blood, saline, etc.) through the end hole 305. In such embodiments can inject a therapeutic into the first port (e.g., FIG. 1, 103). The reflux at the distal tip of the catheter minimizes antegrade flow of the therapeutics into the distal normal vessels while inducing preferential flow into the tumor-supplying vessels. Reflux-based embodiments can be useful for normal tissues that are susceptible to ischemic damage even with temporary stoppage of perfusion, such as brain.

As noted, some embodiments utilize an endovascular coil. Endovascular coils are typically used to block blood flow into diseased or injured vessels. However, such coils can also be used to temporarily occlude a blood vessel. Endovascular coils are generally categorized as microcoils (diameters of approximately 0.001 inches to approximately 0.018 inches or approximately 0.025 mm to 0.457 mm), standard coils (diameters of approximately 0.018 inches to approximately 0.038 inches or approximately 0.457 mm to 0.965 mm), and macrocoils (diameters of greater than approximately 0.038 inches or greater than approximately 0.965 mm). Endovascular coils of some preferred embodiments can range in size from approximately 0.001 inches to approximately 0.028 inches (or approximately 0.025 mm to approximately 0.711 mm). Endovascular coils used in various embodiments may be manufactured by one or more of Medtronic, Styker Corporation, Terumo Corporation, Cook Medical, MicroVention, Penumbra, Inc., and/or any other manufacturer of endovascular coils. A common unit for catheters is the French (Fr or F); one of skill in the art will understand that certain embodiments select an endovascular coil based on the size in Frenches versus a metric or Imperial unit (i.e., mm, cm, inches, etc.). The shaft is used to push the coil outside of the end hole to induce temporary occlusion into the normal tissue supplying vessels to induce flow diversion. After completion of therapeutics infusion, the shaft is used to pull the coil back into the end hole.

Additionally or alternatively, certain embodiments utilize a balloon as an occlusion mechanism. Balloon catheters have been used for various purposes including for dilation, occlusion, and delivery of certain medical devices (e.g., stents, prosthetic valves, etc.). Balloon catheters consist of a balloon at the distal end, a long, hollow shaft and insufflation port at the proximal end. The balloon shaft is used to push and pull the distal balloon. The insufflation port is used to inflate or deflate the balloon. Typical insufflation fluids include saline and/or a contrast agent. The balloon can be in its deflated state when introduced into the end hole port 104, through the second lumen 109 and exist the end hole 106 and appropriately positioned within the vessel supplying normal tissue. After confirming the appropriate positioning of the balloon, the balloon can be insufflated by injecting a fluid (e.g., saline, contrast agent, gas, etc.) into an insufflation port. Once insufflated, the balloon can occlude the vessel and divert the blood flow away from the vessel distal to it. Therapeutics injected into the first port (FIG. 1, 103) preferentially go into the tumor supplying vessel, as shown in FIG. 3B. After completing delivering therapeutics, the balloon can then deflated by aspirating fluid or gas from the insufflation port of the balloon catheter and the balloon catheter can be removed.

In many of these embodiments, the shaft diameter for a balloon can range in size from approximately 0.001 inches to approximately 0.028 inches (or approximately 0.025 mm to approximately 0.711 mm) or the Fr equivalent thereof. Additionally, the insufflated diameter of the balloon can be of any size that is sufficient to occlude the downstream vasculature. In various instances, the size can range from approximately 0.5 mm to approximately 10 mm, including 0.5 mm, 0.75 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm.

Additionally or alternatively, certain embodiments utilize a plug as an occluding mechanism. Similar to the balloon described above, the plugs can be connected to a stiff shaft, allowing a plug to be pushed or pulled through the catheter. The plugs may be introduced into the second port 104. Plugs in various embodiments can be made of various materials that enable them to conform to the catheter lumen while they are passing through the catheter. Once a plug exits the end hole 106, it can expand to its original shape and diameter to occlude a blood vessel. As shown in FIG. 3B, the plug can occlude the vessel and divert the blood flow away from the vessel distal to it. Therapeutics injected into the first port (FIG. 1, 103) preferentially go into the tumor supplying vessel, as shown in FIG. 3B. After completing the delivery of therapeutics, the plug can be retracted into the catheter body, so that normal blood flow can be restored.

A plug may be constructed of a pliable or resilient material that can allow for the plug to be retracted to within a lumen for easier withdrawal of a catheter 100. Such materials can include various polymeric materials, fabric materials, and/or certain metallic materials. Examples of polymeric materials can include one or more of polyethylene terephthalate (PET), polyurethane, polyurethane-polycarbonate matrix, expanded polytetrafluoroethylene (ePTFE), and/or any other biocompatible polymer. Exemplary cloth materials include woven fabrics manufactured of one or more of nylon, dacron, polyester, and/or any other natural or synthetic biocompatible fabric. Metallic materials can include a materials with some amount of flexibility and/or have a shape memory ability. Biocompatible metallic materials can include stainless steel, nitinol, and/or any other biocompatible metallic material. The deployed plug size (i.e., diameter) can be of any size that is sufficient to occlude the downstream vasculature. In various instances, the size can range from approximately 0.5 mm to approximately 10 mm, including 0.5 mm, 0.75 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, or 10 mm. As with the balloon described above, the shaft diameter for the plug can range in size from approximately 0.001 inches to approximately 0.028 inches (or approximately 0.025 mm to approximately 0.711 mm) or the Fr equivalent thereof.

Certain plugs as described above can include tissue engaging elements to secure a plug's position. Such tissue engaging elements can take various forms, including pins, barbs, or other form of anchor. Descriptions of such elements can be found in U.S. Pat. No. 11,517,428; the disclosure of which is hereby incorporated by reference in its entirety.

As noted previously, a purpose of catheters described herein can deliver a therapeutic, including Y90 microspheres, to a tumor. In many instances, the therapeutic is delivered via a lumen other than the lumen used for an occlusion mechanism. In many instances, the lumen for the therapeutic terminates at a side port 105. In some instances, a single lumen may be shared between the therapeutic and the occlusion mechanism. This will require a specially designed Y-shape connector (FIG. 4) without dead space, which will be described below.

Various instances may utilize one or both of an internal and an external mechanism to help guide or steer a catheter 100. Mechanisms to guide or steer a catheter include (but are not limited to) a guidewire, magnetic navigation, a steerable tip, and/or any other relevant mechanism to steer a catheter into position. Most commonly, a guidewire in the second (end-hole) lumen 109 will be used to navigate the catheter.

Certain embodiments incorporate a softer or thinner material at distal tip 107. By using a softer or thinner material, distal tip 107 may be less damaging to the vascular endothelium. Additionally, the distal tip 107 can be tapered to minimize potential trauma to the vessel wall when advancing the catheter over a guidewire. To assist with viewing the position of the catheter, the distal tip 107 may further include a radiopaque material that can be seen by a fluoroscopic imaging. Such materials can be one or more metallic rings and/or a particular patterning of metallic materials. In some instances the radiopaque material is a platinum-iridium marker. In certain instances, the radiopaque material is a polymer doped with bismuth or barium sulfate (BaSO₄).

Some embodiments also include a lubricous or hydrophilic material covering the catheter body 102, such as a sleeve or coating. A lubricous or hydrophilic material can reduce friction with the vascular endothelium to assist with positioning of the catheter and/or reduce the possibility of damage to the vascular endothelium. In many instances, the lubricous material is a hydrogel.

### Dead-Space-Free Y-Adapter

As noted above, an alternative to a dual-lumen catheter includes a single lumen catheter with a dead-space-free Y-adapter 400, which can also achieve distal protection and flow diversion. In such embodiments, a space formed between the shaft of the occlusion mechanism and the outer wall of the single lumen catheter serves as a lumen for the delivery of therapeutics. The Y-adapter 400 can be used to provide two introduction ports: one for an occlusion device and the other for the delivery of therapeutics.

As noted previously, a Tuohy-Borst adaptor could provide a similar functionality, but retrograde pooling can cause a therapeutic to remain within a dead space of commercially available Y-adapters. Thus, such adapters are not suitable to be used in the setting of delivery of therapeutic as well as distal protection and flow diversion. In addition to wasting an amount of the therapeutic, this retention can also be harmful to a physician or medical practitioner when using a radiotherapeutic (e.g., Y90).

FIGS. 4A-4B provide illustrations of a dead-space-free Y-adapter 400 which can prevent retrograde pooling of therapeutics. In FIG. 4A, the Y-shaped adapter 400 having a hollow body forming a linear section (e.g., linear conduit) 402 that can be used for the introduction of an occlusion device and one side branch (e.g., branch conduit) 406 that is axially diverging from the linear section 402 at a branch point and can be used for injection of therapeutics.

Many instances include a gasket or hemostatic seal 408 located near the branching point of the Y-shaped adapter 400. The hemostatic seal 408 can form an opening or pore to allow a wire or a shaft (e.g., shaft for a plug or balloon) and seal the branch conduit 406, thereby preventing any retrograde flow or therapeutic stagnation within the Y-adapter. The hemostatic seal 408 can be held in place by conforming to a molded shape within the Y-shaped adapter 400. As illustrated, in some instances, the hemostatic seal 408 is conically shaped to match a conical constriction within the Y-shaped adapter 400. In some instances, a rigid washer may be placed one or more ends of the hemostatic seal 408 to provide a compression pressure on the hemostatic seal 408 to ensure a tight seal between the hemostatic seal 408, a shaft passing through the hemostatic seal 408, and the Y-shaped adapter 400.

Additional embodiments incorporate a locking assembly 410 in the port for the linear conduit 402 of the Y-shaped adapter 400. The locking assembly 410 has a central hole that matches the pore within the hemostatic seal 408 to allow a guidewire or a shaft to pass through the locking assembly and the hemostatic seal 408. Such locking assembly 410 can interact with a locking mechanism (e.g., Luer) to provide the compression pressure on the hemostatic seal 408 or a washer positioned on the hemostatic seal 408. This compressive pressure can also lock and/or immobilize a guidewire or a shaft by placing additional pressure on them. FIG. 4A illustrates the locking mechanism 410 in an unlocked position, where it applies no pressure against the hemostatic seal 408.

A Y-shaped adapter 400 can further include a joining mechanism 412 to connect with a catheter, as shown in FIG. 4B. In such instances, the Y-shaped adapter 400 can be added to an existing single-lumen catheter hub 409. The joining mechanism 412 can connect the Y-shaped adapter 400 to a catheter permanently or reversibly. Permanent methods can include an adhesive, contact cement, swaging, and/or any other permanent method. Reversible methods include screwing or twisting methods, such as a Luer lock, mated threading, and/or any other reversible method.

FIG. 4B illustrates a Y-shaped adapter 400 mounted on a catheter 450. As illustrated in FIG. 4B, an occlusion mechanism 452 can be attached to a shaft 451, which passes through the linear section 402, the locking assembly 410, the hemostatic seal 408, and the catheter 450. This shaft 451 is connected to the occlusion mechanism 452 at the distal end of the device.

As shown in FIG. 4B, the locking mechanism 410 is in the locked position to apply pressure to the hemostatic seal 408. In this position, retrograde flow from the side port 406 (e.g., flow into linear section 402) can be prevented and the occlusion mechanism 452 and its shaft 451 is also locked in place in relation to catheter 450.

As shown in FIG. 4C, once the distal occlusion device 452 is delivered through the single-lumen catheter into the normal-tissue supplying vessel, the single lumen catheter 450 is pulled back so that its end hole 453 is in the vicinity to the tumor vessels. The occlusion device 452 is connected to a thin shaft 451 that is used to place, reposition, and retrieve the occlusion device 452.

### Methods of Delivering a Therapeutic

Many embodiments can be used to deliver a therapeutic (e.g., Y90 microspheres) to a tumor. In many such instances, a catheter is inserted into an individual. In various instances, the individual is a mammal. In various instances, the mammal is a non-human mammal, a human, a primate, a household animal (e.g., dog, cat, bird, guinea pig, etc.), livestock or farm animal (e.g., cow, pig, horse, chicken, goat, sheep, etc.). In certain instances, the individual is a cadaver. In certain instances, the individual is a medical analog, such a dummy, a model, an anthropomorphic phantom, and/or other simulant.

In many instances, the catheter is inserted into the vasculature of the individual. Such vasculature can be venous or arterial. In various instances, the vasculature is accessed via a groin or a wrist. In certain instances, an artery feeding the target tissue is accessed directly. In other instances, a different artery is accessed, where the catheter is maneuvered retrograde through the accessed artery toward the aorta, until an artery feeding the target tissue or tumor is accessed. The catheter can be maneuvered until it is in the desired position to deliver the therapeutic. As noted above, catheters of many embodiments are used to deliver Y90 microspheres to a liver of an individual—in such instances, the hepatic artery is the desired artery to access. Maneuvering a catheter can utilize image guidance, such as fluoroscopy, MRI guidance, ultrasound-based guidance, electroanatomical mapping, CT guidance, and/or any other method for image guidance.

A desired position for the catheter is when the distal tip is in the vicinity of the tumor. Depending on the particular embodiment, the distal tip may be placed upstream, downstream, and/or at artery feeding a tumor. In many instances, the catheter is placed, such that the port that is used to deliver the therapeutic is upstream of the tumor vasculature.

Once in position, the occlusion mechanism can be deployed. As described above, deployment can involve pushing an endovascular coil, balloon, and/or plug out of the end hole of the catheter. Endovascular coils and plugs are occlusive when positioned within vessels. Balloons, once positioned, can be insufflated to achieve vessel occlusion. Balloon catheters have insufflation ports at the proximal end so that operator can insufflate the balloons by injecting liquid or gas. FIG. 3A illustrates a catheter 300 after deployment of an endovascular coil 303 that occludes branching vasculature. A similar strategy can be used to deploy an occlusion mechanism into a non-branched vasculature. With this strategy, proximal port 302 is positioned at or slightly upstream from the vasculature feeding the tumor. When using a balloon, the insufflation fluid can be injected into an appropriate port, so the balloon occludes the downstream vasculature.

FIG. 3C illustrates another strategy that uses an injection port on the control hub to perfuse saline and/or blood to minimize antegrade flow. In such embodiments, the saline and/or blood 305 exits the catheter via a distal port. An increased flow or pressure caused by saline and/or blood minimizes the antegrade flow of the non-perfused blood. A therapeutic can then be distributed using a proximal port that is located upstream of the tumor.

Once an occlusion mechanism is deployed, a therapeutic may be injected using an injection port on a control hub. The therapeutic may be of any variety described herein, including a chemotherapeutic, radiotherapeutic, etc. In some preferred embodiments, the therapeutic comprises Y90 microspheres. As noted above, the therapeutic may exit out of a proximal port and/or distal port, depending on a particular catheter and/or occlusion mechanism configuration. Once the full amount of the therapeutic is injected, some instances flush the catheter with saline, blood, or another fluid. The flushing can ensure that all or nearly all of the therapeutic is delivered to the tumor. In some instances (e.g., when using Y90 microspheres or another radiotherapeutic), flushing the catheter can minimize residual radiation within catheter, which may be beneficial for disposal of the catheter.

Further embodiments remove the occlusion mechanism from the downstream vasculature. When using a balloon, pressure may be removed from the balloon and/or the insufflation is removed from the balloon. In many situations, the occlusion mechanism and/or a shaft connected to the occlusion mechanism is retracted into the catheter body. Such retraction can involve pulling an occlusion mechanism on the control hub, such that the plug, balloon, or coil retract within the catheter body.

The catheter may be retracted through the body by any relevant method, such as gently pulling the catheter from its entry point into the body. Once the catheter is fully removed, the entry point may be sealed using an appropriate means to achieve hemostasis. The sealing method may include one or more of allowing the wound to clot, sutures, staples, an adhesive, and/or any other appropriate method. The entry point can then be bandaged or otherwise covered.

For purposes of completeness, various aspects of the present disclosure are set out in the following numbered clauses.
Aspect 1. A device to deliver a therapeutic, the device comprising:
a hub comprising two ports, wherein a first port is configured to accept a bolus of Y90 beads and the second port is configured to accept an occlusion mechanism;
a catheter body formed as generally cylindrical body spanning from a proximal end and a distal end and forming two lumens and two exit holes, wherein the proximal end adjoins the hub, such that the first port is in fluid communication with a first lumen and a first exit hole, and the second port is in fluid communication with a second lumen and a second exit hole, wherein the second exit hole is located at the distal end of the catheter body and the first exit hole located at a distance d proximal from the distal end; and
an occlusion mechanism disposed within the second lumen.
   Aspect 2. The device of Aspect 1, wherein the distance d is from 0 cm to approximately 10 cm.
   Aspect 3. The device of Aspect 1 or 2, wherein the occlusion mechanism is selected from an endovascular coil, a plug, and a balloon catheter.
   Aspect 4. The device of any one of Aspects 1-3, wherein the hub is formed as a linear body with opposing proximal and distal ends and comprising an axially diverging branch, wherein the distal end of the control hub adjoins the proximal end of the elongated body, wherein the first port is positioned on the axially diverging branch, and wherein the second port is positioned on the proximal end of the hub.
   Aspect 5. The device of any one of Aspects 1-4, wherein the elongated body possesses an outer diameter of approximately 1-5 Fr.
   Aspect 6. The device of any one of Aspects 1-5, wherein the therapeutic is selected from a chemotherapeutic, a radiotherapeutic, and a biological agent.
   Aspect 7. The device of any one of Aspects 1-6, wherein the therapeutic is a radiotherapeutic.
   Aspect 8. The device of any one of Aspects 1-7, wherein the therapeutic comprises yttrium-90 microspheres.
   Aspect 9. A device to deliver Y90 to a tumor, comprising:
a catheter body formed as a tube having opposing proximal and distal ends and defining a first lumen and a second lumen;
an occlusion mechanism disposed within the second lumen; and
an injection port in fluid communication with the first lumen and configured to receive a bolus of Y90 beads.
   Aspect 10. The device of Aspect 9, wherein the occlusion mechanism is selected from a balloon catheter, vascular plug, and an endovascular coil.
   Aspect 11. The device of Aspect 9 or 10, further comprising a hub adjoining the proximal end of the catheter body, wherein the injection port is comprised within the hub, and wherein the hub further forms a port configured to receive the occlusion mechanism.
   Aspect 12. The device of any one of Aspects 9-11, wherein the hub is formed as a linear body with opposing proximal and distal ends and comprising an axially diverging branch, wherein the distal end of the control hub adjoins the proximal end of the catheter body, wherein the port configured to receive the occlusion mechanism is located at the proximal end of the control hub, and wherein the axially displaced branch forms the injection port.
   Aspect 13. The device of any one of Aspects 9-12, wherein the catheter body possesses an outer diameter of approximately 1-5 Fr.
   Aspect 14. A method of delivering Y90 beads to an individual, comprising
occluding downstream vasculature in an individual;
providing a dose of Y90 beads to tumor vasculature in the individual, wherein the tumor vasculature is located upstream of the downstream vasculature.
   Aspect 15. The method of Aspect 14, further comprising navigating a device of any one of Aspects 1-13 through the vasculature of an individual, such that the distal end of the device is located upstream of the tumor vasculature.
   Aspect 16. The method of Aspect 15, wherein occluding the downstream vasculature comprises insufflating a balloon, deploying a plug or a coil occluder.
   Aspect 17. The method of Aspect 14, further comprising navigating a device of any one of Aspects 15-20 through the vasculature of an individual, such that the distal end of the device is located downstream of the tumor vasculature
   Aspect 18. The method of any one of Aspects 14-17, wherein providing a dose of Y90 beads comprises injecting the dose of Y90 beads into the injection port.
   Aspect 19. The method of any one of Aspects 14-18, wherein an angle formed between the downstream vasculature and the tumor vasculature is greater than 90°.
   Aspect 20. A Y adapter, comprising:
a hollow body having a general Y-shape forming a linear conduit and a branch conduit axially diverging from the linear conduit at a branch point;
a hemostatic seal located at the branch point and forming a pore to allow an occlusion mechanism to pass through the hemostatic seal but prevents retrograde flow in the linear conduit.
   Aspect 21. The Y adapter of Aspect 20, further comprising a locking mechanism configured to apply an axial pressure on the hemostatic seal.
   Aspect 22. The Y adapter of Aspect 21, further comprising a Luer lock disposed on the linear conduit that can interact with the locking mechanism to apply an axial pressure on the hemostatic seal.
   Aspect 23. The Y adapter of Aspect 21 or 22, wherein the locking mechanism forms a pore configured to allow an occlusion mechanism to pass through the locking mechanism.
   Aspect 24. The Y adapter of Aspect 23, wherein the pore on the locking mechanism aligns with the pore on the hemostatic seal.
   Aspect 25. The Y adapter of any one of Aspects 20-24, further comprising a joining mechanism configured to attach the hollow body to a catheter.
   Aspect 26. A catheter, comprising:
a catheter body forming a central lumen and extending between a proximal end and a distal end; and
a Y adapter of any one of Aspects 20-25 adjoined to the proximal end of the catheter body.

### Exemplary Embodiments

Although the following embodiments provide details on certain embodiments of the inventions, it should be understood that these are only exemplary in nature and are not intended to limit the scope of the invention.

### Example 1: Occluding Downstream Flow

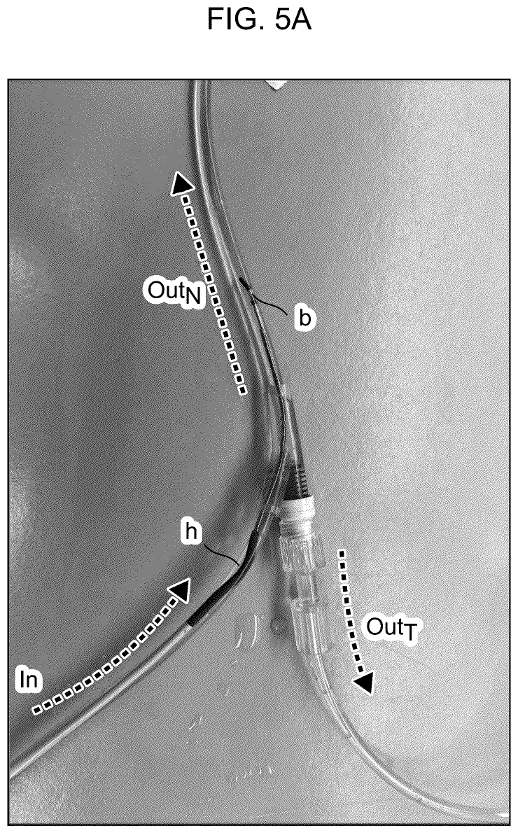

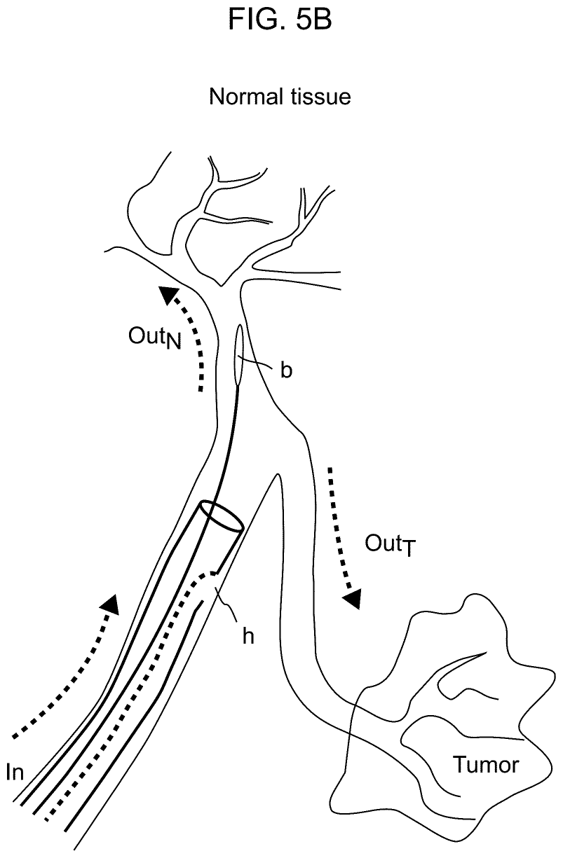

This example provides a proof of concept using tubing as a vascular analog. FIG. 5A illustrates the tubing with a connector creating a Y-shaped analog. The blood analog flows from the Inflow (In) to the Outflow (Out_{T} and Out_{N}), where Out_{T} represents the flow of blood into a tumor, while Out_{N} is normal outflow to downstream, healthy tissue. FIG. 5B illustrates a representation of the vasculature demonstrated in the analog of FIG. 5A. FIGS. 5A-5B also illustrate the positioning of a catheter into the vasculature just proximal to the tumor vasculature (Out_{T}). Furthermore, an occluding balloon is placed in the normal vasculature (Out_{N}), slightly downstream of the tumor vasculature. In this illustration, a balloon (b) is used to temporarily occlude the normal or healthy vasculature Out_{N}, while side exit hole h is positioned upstream of the tumor vasculature.

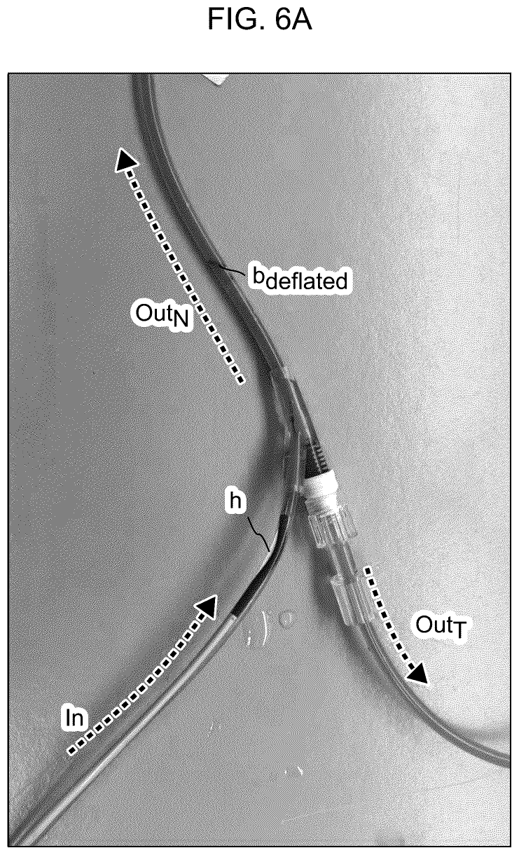

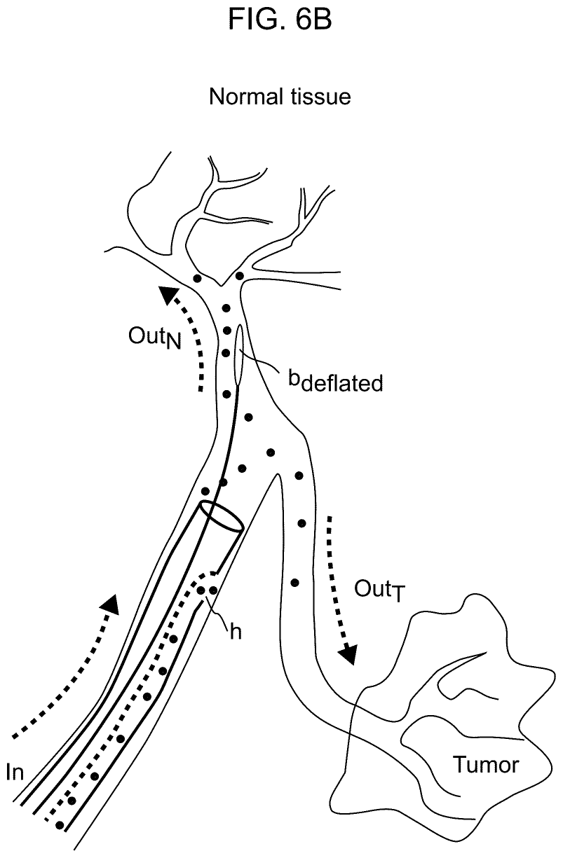

FIGS. 6A-6B provide a demonstration of flow through the system using a dye, where the dye follows the path illustrated in FIGS. 5A-5B. In the present example the dye was perfused through the system flows unimpeded into both outflow vessels towards tumor (Out_{T}) and towards normal tissue (Out_{N}). As illustrated in FIGS. 6A-6B, the introduction of any fluid into the system via a catheter will flow to both healthy and tumor tissue, as the uninflated balloon (b_{deflated}) does not occlude the healthy vasculature Out_{N}.

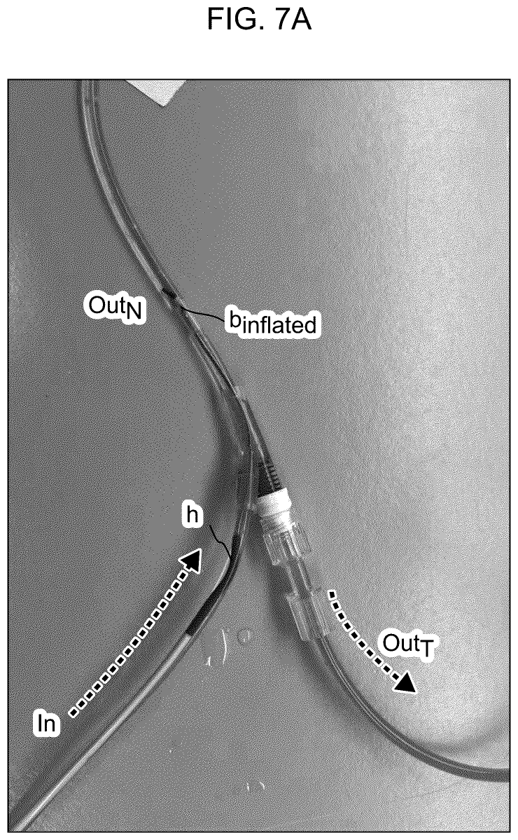

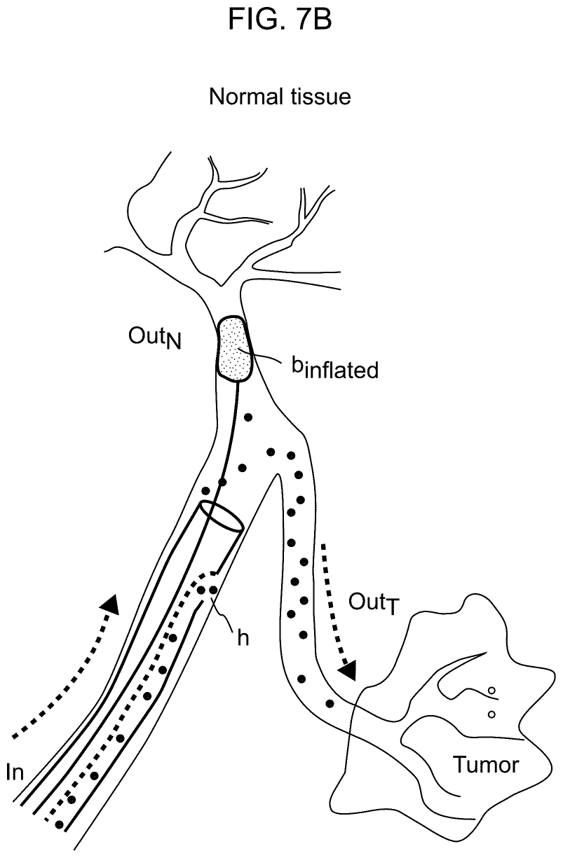

FIGS. 7A-7B illustrate reversible occlusion of the normal vasculature (Out_{N}) by insufflating the balloon (b_{inflated}) to occlude the normal vasculature Out_{N}. This occlusion causes the dye to exclusively enter the tumor vasculature (Out_{T}). In this situation, Y90 beads would enter the tumor without sacrificing healthy tissue.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A device to deliver a therapeutic, the device comprising:
a hub comprising two ports, wherein a first port is configured to accept a bolus of a therapeutic and the second port is configured to accept an occlusion mechanism;
a catheter body formed as generally cylindrical body spanning from a proximal end and a distal end and forming two lumens and two exit holes, wherein the proximal end adjoins the hub, such that the first port is in fluid communication with a first lumen and a first exit hole, and the second port is in fluid communication with a second lumen and a second exit hole, wherein the second exit hole is located at the distal end of the catheter body and the first exit hole located at a distance d proximal from the distal end; and
an occlusion mechanism disposed within the second lumen.

2. The device of claim 1, wherein the distance d is from 0 cm to approximately 10 cm.

3. The device of claim 1, wherein the occlusion mechanism is selected from an endovascular coil, a plug, and a balloon catheter.

4. The device of claim 1, wherein the hub is formed as a linear body with opposing proximal and distal ends and comprising an axially diverging branch, wherein the distal end of the control hub adjoins the proximal end of the elongated body, wherein the first port is positioned on the axially diverging branch, and wherein the second port is positioned on the proximal end of the hub.

5. The device of claim 1, wherein the elongated body possesses an outer diameter of approximately 1-5 Fr.

6. The device of claim 1, wherein the therapeutic is selected from a chemotherapeutic, a radiotherapeutic, and a biological agent.

7. The device of claim 1, wherein the therapeutic is a radiotherapeutic.

8. The device of claim 1, wherein the therapeutic comprises yttrium-90 microspheres.

9-13. (canceled)

14. A method of delivering Y90 beads to an individual, comprising
occluding downstream vasculature in an individual;
providing a dose of Y90 beads to tumor vasculature in the individual, wherein the tumor vasculature is located upstream of the downstream vasculature.

15. The method of claim 14, further comprising navigating a device to deliver a therapeutic through the vasculature of an individual, such that the distal end of the device is located upstream of the tumor vasculature, wherein the device to deliver a therapeutic comprises:
a hub comprising two ports, wherein a first port is configured to accept a bolus of a therapeutic and the second port is configured to accept an occlusion mechanism;
a catheter body formed as generally cylindrical body spanning from a proximal end and a distal end and forming two lumens and two exit holes, wherein the proximal end adjoins the hub, such that the first port is in fluid communication with a first lumen and a first exit hole, and the second port is in fluid communication with a second lumen and a second exit hole, wherein the second exit hole is located at the distal end of the catheter body and the first exit hole located at a distance d proximal from the distal end; and
an occlusion mechanism disposed within the second lumen.

16. The method of claim 15, wherein occluding the downstream vasculature comprises insufflating a balloon, deploying a plug or a coil occluder.

17. The method of claim 14, further comprising navigating a device to deliver a therapeutic through the vasculature of an individual, such that the distal end of the device is located downstream of the tumor vasculature, wherein the device to deliver a therapeutic comprises:
a hub comprising two ports, wherein a first port is configured to accept a bolus of a therapeutic and the second port is configured to accept an occlusion mechanism;
a catheter body formed as generally cylindrical body spanning from a proximal end and a distal end and forming two lumens and two exit holes, wherein the proximal end adjoins the hub, such that the first port is in fluid communication with a first lumen and a first exit hole, and the second port is in fluid communication with a second lumen and a second exit hole, wherein the second exit hole is located at the distal end of the catheter body and the first exit hole located at a distance d proximal from the distal end; and
an occlusion mechanism disposed within the second lumen.

18. The method of claim 14, wherein providing a dose of Y90 beads comprises injecting the dose of Y90 beads into the injection port.

19. The method of claim 14, wherein an angle formed between the downstream vasculature and the tumor vasculature is greater than 90°.

20. A Y adapter, comprising:
a hollow body having a general Y-shape forming a linear conduit and a branch conduit axially diverging from the linear conduit at a branch point;
a hemostatic seal located at the branch point and forming a pore to allow an occlusion mechanism to pass through the hemostatic seal but prevents retrograde flow in the linear conduit.

21. The Y adapter of claim 20, further comprising a locking mechanism configured to apply an axial pressure on the hemostatic seal.

22. The Y adapter of claim 21, further comprising a Luer lock disposed on the linear conduit that can interact with the locking mechanism to apply an axial pressure on the hemostatic seal.

23. The Y adapter of claim 21, wherein the locking mechanism forms a pore configured to allow an occlusion mechanism to pass through the locking mechanism.

24. The Y adapter of claim 23, wherein the pore on the locking mechanism aligns with the pore on the hemostatic seal.

25. The Y adapter of claim 20, further comprising a joining mechanism configured to attach the hollow body to a catheter.

26. (canceled)

27. The device of claim 4, wherein the axially diverging branch diverges from the linear body at a branch point, and further comprising:
a hemostatic seal located at the branch point and forming a pore to allow an occlusion mechanism to pass through the hemostatic seal but prevents retrograde flow in the linear conduit.

28. The device of claim 27, further comprising a locking mechanism configured to apply an axial pressure on the hemostatic seal.

29. The device of claim 28, further comprising a Luer lock disposed on the linear conduit that can interact with the locking mechanism to apply an axial pressure on the hemostatic seal.

30. The device of claim 28, wherein the locking mechanism forms a pore configured to allow an occlusion mechanism to pass through the locking mechanism.

31. The device of claim 30, wherein the pore on the locking mechanism aligns with the pore on the hemostatic seal.

32. The device of claim 27, further comprising a joining mechanism configured to attach the hollow body to the catheter body.
